(19) United States
(12) Patent Application Publication
(10) Pub. No.: US 2024/0175871 A1
(43) Pub. Date: May 30, 2024

(54) ALPHA-1-ANTITRYPSIN PHENOTYPE C DETECTION AND TREATMENT OF INFLAMMATION AND INFLAMMATORY CONDITIONS ASSOCIATED WITH SAID PHENOTYPE

(71) Applicant: Kevin F. LAW, Yardley, PA (US)

(72) Inventor: Kevin F. LAW, Yardley, PA (US)

(21) Appl. No.: 18/517,463

(22) Filed: Nov. 22, 2023

Related U.S. Application Data
(60) Provisional application No. 63/428,396, filed on Nov. 28, 2022.

(51) Int. Cl. G01N 33/569; A61K 38/57

(52) U.S. Cl. CPC G01N 33/56983; A61K 38/57; G01N 2333/165; G01N 2333/8125; G01N 2469/10

(57) ABSTRACT

A method of treating a patient infected with SARS-COV-2 virus includes phenotyping a sample obtained from the patient; and administering alpha-1-antitrypsin to the patient only if an alpha-1-antitrypsin C phenotype is detected in the sample. A kit configured to detect a SARS-COV-2 infection and an alpha-1-antitrypsin C phenotype includes a first sample collection container for holding a sample to be tested for a SARS-COV-2 infection; and a second sample collection container for holding a sample to be tested for alpha-1-antitrypsin phenotype C.

### CROSS-REFERENCE TO RELATED APPLICATIONS

This application claims the benefit under 35 U.S.C. § 119(e) of U.S. Provisional Patent App. No. 63/428,396, filed Nov. 28, 2022, the contents of which application are incorporated herein by reference in their entireties for all purposes.

### BACKGROUND OF THE INVENTION

### 1. Field of Invention

This invention relates to a method for detecting an alpha-1-antitrypsin phenotype and to a method of treating a condition affected by said phenotype.

### 2. Description of Related Art

Alpha-1-antitrypsin (“AAT”) is a serine protease inhibitor, which is found in human serum and other fluids. AAT plays an important role in the regulation of tissue proteolysis. It is an inhibitor of neutrophil elastase, and protects the lungs from proteolytic damage (1).

AAT testing is primarily reserved for testing of patients with COPD, asthma with fixed airflow obstruction, and bronchiectasis as part of the so-called GOLD recommendations. The purpose of this is to primarily identify the rare (roughly 1/4000) doubly deficient ZZ patients who develop emphysema or bronchiectasis in an accelerated fashion. Roughly 95% of the US population is MM with the other 5% representing heterozygotes that bear a point mutation like MS, MZ and to a much lesser extent MF, MV, and MX. In a COPD population, there is some enrichment of MZ because it is recognized as a distinct “endotype” of COPD with a relative deficiency of AAT. Since the Z allele also confers a risk for liver disease, gastroenterologists will check for deficiency in the setting of cirrhosis or unexplained liver disease. A rare disorder, panniculitis, will also precipitate testing as it is associated with the ZZ genotype. There are a variety of rare phenotypes, with “C” being one, for which little is known except that they do not appear to be associated with a change in the genotype.

Over 40 AAT phenotypes have been identified, including the C phenotype. The C phenotype is a finding predictive of poor host response to neutrophilic inflammation. It identifies patients at risk for higher mortality, past or present cancer, and acute and chronic inflammatory conditions.

Since 95% of most populations are MM genotype, it is most often expressed as CM, but can be expressed as CS or CZ for the two most common deficiency alleles. The CM and CS phenotypes correlate with MM and MS genotypes, suggesting post-translational modification. Individuals identified as CM or CS have characteristic C4 and C6 bands anodal and cathodal to the M2 band in immunoelectrophoresis, which were associated with Active Cancer (Odds Ratio 9.26) and, two-year Mortality (Odds Ratio 11.5) (2).

AAT inhibits entry of SARS-COV-2 into cells by inhibiting protease mediated activation of the viral spike protein necessary for binding to ACE2. Specifically, AAT inhibits the transmembrane protease TMPRSS2 which has been described as a principal mode of entry into host cells (3,4).

A relative deficiency in AAT may contribute to disease severity as IL-6:AAT ratios have been correlated with ICU utilization and mortality in COVID infection (5).

AAT has been proposed as a potential therapy for severe COVID infection (6,7).

All references cited herein are incorporated herein by reference in their entireties.

### BRIEF SUMMARY OF THE INVENTION

Accordingly, a first aspect of the invention is a method of treating a patient infected with SARS-COV-2 virus, said method comprising: phenotyping a sample obtained from the patient; and administering alpha-1-antitrypsin to the patient only if an alpha-1-antitrypsin C phenotype is detected in the sample, wherein the alpha-1-antitrypsin is effective to treat the patient infected with the SARS-COV-2 virus.

In certain embodiments, the phenotyping comprises the sequential steps of: separating proteins in the sample by isoelectric focusing; incubating the sample with peroxidase labeled anti-alpha-1-antitrypsin antisera; removing unbound proteins; staining bound proteins; and reading a resulting banding pattern to determine whether the patient has the alpha-1-antitrypsin C phenotype.

In certain embodiments, the alpha-1-antitrypsin is administered intravenously.

In certain embodiments, the alpha-1-antitrypsin is first administered to the patient while the patient has an oxygen saturation level of at least 94%.

In certain embodiments, the method further comprises administering to the patient at least one member selected from the group consisting of Baricitinib, Remdesivir, Nirmatrelvir, Ritonavir, Bebtelovimab and Molnupiravir.

In certain embodiments, the method further comprises detecting whether the patient is infected with SARS-COV-2 virus.

In certain embodiments, the alpha-1-antitrypsin C phenotype is CM, CS or CZ phenotype.

A second aspect of the invention is a kit configured to detect a SARS-COV-2 infection and an alpha-1-antitrypsin C phenotype, said kit comprising: a first sample collection container for holding a sample to be tested for a SARS-COV-2 infection; and a second sample collection container for holding a sample to be tested for alpha-1-antitrypsin phenotype C.

In certain embodiments, the kit further comprises an antigen-binding moiety effective to specifically bind to a SARS-COV-2 antigen.

In certain embodiments, the kit further comprises a SARS-COV-2 specific antibody and a control antibody immobilized on two distinct locations of a substrate.

### BRIEF DESCRIPTION OF SEVERAL VIEWS OF THE DRAWINGS

The invention will be described in conjunction with the following drawings in which like reference numerals designate like elements and wherein:

FIG. 1 and FIG. 2 are photographs of immunoelectrophoresis gels.

### DETAILED DESCRIPTION OF PREFERRED EMBODIMENTS OF THE INVENTION

### Definitions

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to an “antibody” is a reference to one or more antibodies, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

The meaning of certain terms and phrases employed in the specification, examples, and appended claims are provided. The definitions are not meant to be limiting in nature and serve to provide a clearer understanding of certain aspects of the present invention.

As used herein, the term “antigen” is generally used in reference to any substance that is capable of reacting with an antibody. More specifically, as used herein, the term “antigen” refers to a synthetic peptide, polypeptide, protein or fragment of a polypeptide or protein, or other molecule which elicits an antibody response in a subject, or is recognized and bound by an antibody.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); or that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

The terms “patient,” “individual,” or “subject” are used interchangeably herein, and refer to a mammal, particularly, a human. The patient may have a mild, intermediate or severe disease or condition. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining or providing a patient sample and/or detecting the level of biomarker(s) in a sample. In one embodiment, the terms refer to obtaining or providing a patient sample and detecting the level of one or more biomarkers in the sample. In another embodiment, the terms “measuring” and “determining” mean detecting the level of one or more biomarkers in a patient sample. The term “measuring” is also used interchangeably throughout with the term “detecting.” In certain embodiments, the term is also used interchangeably with the term “quantitating.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of a disease such as COVID-19. Moreover, a sample obtained from a patient can be divided and only a portion may be used for diagnosis. Further, the sample, or a portion thereof, can be stored under conditions to maintain sample for later analysis. The definition encompasses liquid samples of biological origin (including, but not limited to, blood, peripheral blood, serum, plasma, cord blood, amniotic fluid, urine, saliva, stool, mucus, sputum and synovial fluid), solid tissue samples such as a biopsy specimen or tissue cultures or cells derived therefrom and the progeny thereof.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

Various methodologies of the instant invention include a step that involves comparing a value, level, feature, characteristic, property, etc. to a “suitable control,” which may be referred to interchangeably herein as an “appropriate control,” a “control sample,” a “reference” or simply a “control.” A “suitable control,” “appropriate control,” “control sample,” “reference” or a “control” is any control or standard familiar to one of ordinary skill in the art useful for comparison purposes. A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., ELISA, PCR, LC-MS, GC-MS, etc.), where the levels of biomarkers may differ based on the specific technique that is used.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M. As used herein, the terms “specific binding” or “specifically binding” when used in reference to the interaction of an antibody and a protein or peptide means that the interaction is dependent upon the presence of a particular structure (i.e., the epitope) on the protein.

By “antibody” is meant any immunoglobulin polypeptide, or fragment thereof, having immunogen binding ability. As used herein, the terms “antibody fragments”, “fragment”, or “fragment thereof” refer to a portion of an intact antibody. Examples of antibody fragments include, but are not limited to, linear antibodies; single-chain antibody molecules; Fc or Fc′ peptides, Fab and Fab fragments, and multi-specific antibodies formed from antibody fragments. In most embodiments, the terms also refer to fragments that bind an antigen of a target molecule and can be referred to as “antigen-binding fragments.” As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies that specifically binds the target antigen.

The term “conjugate” refers to a complex of two molecules linked together, for example, linked together by a covalent bond. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

The terms “conjugating,” “joining,” “bonding,” “labeling” or “linking” refer to making two molecules into one contiguous molecule; for example, linking two polypeptides into one contiguous polypeptide, or covalently attaching an effector molecule or detectable marker radionuclide or other molecule to a polypeptide. In the specific context, the terms include reference to joining a ligand, such as an antibody moiety, to an effector molecule. The linkage can be either by chemical or recombinant means. “Chemical means” refers to a reaction between the antibody moiety and the effector molecule such that there is a covalent bond formed between the two molecules to form one molecule.

The term “epitope” or “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation. An antigenic determinant can compete with the intact antigen (i.e., the “immunogen” used to elicit the immune response) for binding to an antibody.

By an “effective amount” is meant the amount of a required to ameliorate the symptoms of a disease relative to an untreated patient. The therapeutically effective amount of active compound(s) used to practice the therapeutic method of the invention varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion (e.g., at least about 5, 10, 25, 50, 100, 125, 150, 200, 250, 300, 350, 400, or 500 amino acids or nucleic acids) of a protein or nucleic acid molecule that is substantially identical to a reference protein or nucleic acid and retains at least one biological activity of the reference. In some embodiments the portion retains at least 50%, 75%, or 80%, or more preferably 90%, 95%, or even 99% of the biological activity of the reference protein or nucleic acid described herein.

The terms “isolated,” “purified,” or “biologically pure” refer to material that is free to varying degrees from components which normally accompany it as found in its native state. Various levels of purity may be applied as needed according to this invention in the different methodologies set forth herein; the customary purity standards known in the art may be used if no standard is otherwise specified. Indeed, the term “purified” does not require the material to be present in a form exhibiting absolute purity, exclusive of the presence of other compounds. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

The term “operably linked” means that a first polynucleotide is positioned adjacent to a second polynucleotide that directs transcription of the first polynucleotide when appropriate molecules (e.g., transcriptional activator proteins) are bound to the second polynucleotide.

As used herein, “kit” means a collection of at least two components associated with each other for the purpose of performing at least one particular function, such as, e.g., a diagnostic and/or therapeutic method of the invention. In certain embodiments, one of the at least two components is a set of instructions for using the kit.

### Diagnostic and Therapeutic Methods

The invention is based at least in part on the discovery that the C phenotype of AAT when present in COVID patients predicts increased 30-day and 60-day mortality (odds ratio 4.63 and 4.28 respectively). Since this likely represents AAT MM protein with reduced activity, testing for the C phenotype identifies a unique population of patients that will have improved outcomes with intravenously administered AAT. Thus, the invention encompasses a method for testing for the C phenotype alone or together with testing for SARS-COV-2, and a method for treating COVID-19, wherein patients testing positive for both SARS-COV-2 and the C phenotype are administered AAT.

The C phenotype is expressed with a remarkable frequency in a number of conditions (8) including severe COVID-19, which has distinct implications in terms of prognosis and risk of mortality. These associations suggest the C phenotype as a potential biomarker for such conditions. Since the phenotype appears to be an alteration of the parent common protein, it suggests a change in functionality. Some of the less well-known phenotypes may represent an alteration in the more common parent protein with C being the one most commonly seen in an inpatient setting and in acute inflammatory conditions.

In the treatment method, a sample is obtained from a patient infected with SARS-CoV-2 virus. The sample is analyzed to determine an AAT phenotype. If the C phenotype is detected, AAT is administered to the patient.

Optionally, the method can include a SARS-COV-2 detection step. This step can be conducted separately or in combination with the phenotyping step. The two steps can be conducted simultaneously or in any order. Conducting the virus detection step before the phenotyping step may minimize the number of phenotyping tests conducted on a population. Conducting the phenotyping step before the virus detection step identifies members of the population for whom SARS-COV-2 poses a greater risk so that additional preventative measures might be taken and/or AAT supplementation can be started as soon as a positive SARS-COV-2 test is obtained.

Any current (or future) test for determining whether a patient has the C phenotype is (or will be) suitable for use in the invention. Thus, for example, the C phenotype can be identified via immunoelectrophoresis on agarose gels. See, e.g., Robinet-Lēvy et al. (9) and Greene et al. (10). See also Greene et al. (11), which provides a description of the CM phenotype on IEF agarose gels with C4 and C6 bands anodal and cathodal to the M2 band.

In certain embodiments, the phenotyping step comprises: separating proteins in the sample by isoelectric focusing; incubating the sample with peroxidase labeled anti-alpha-1-antitrypsin antisera; removing unbound proteins; staining bound proteins; and reading a resulting banding pattern to determine whether the patient has the C phenotype.

The C phenotype is not restricted to the MM genotype where it manifests as CM. In the most common deficiency allele genotypes MS and MZ it manifests as CS or CZ. Any disadvantage it confers when expressed by MM genotypes would be expected with heterozygotes with deficiency alleles. Since it appears to suggest decreased functionality in AAT, the disadvantage may be even more apparent in those individuals who are additionally deficient in this protein.

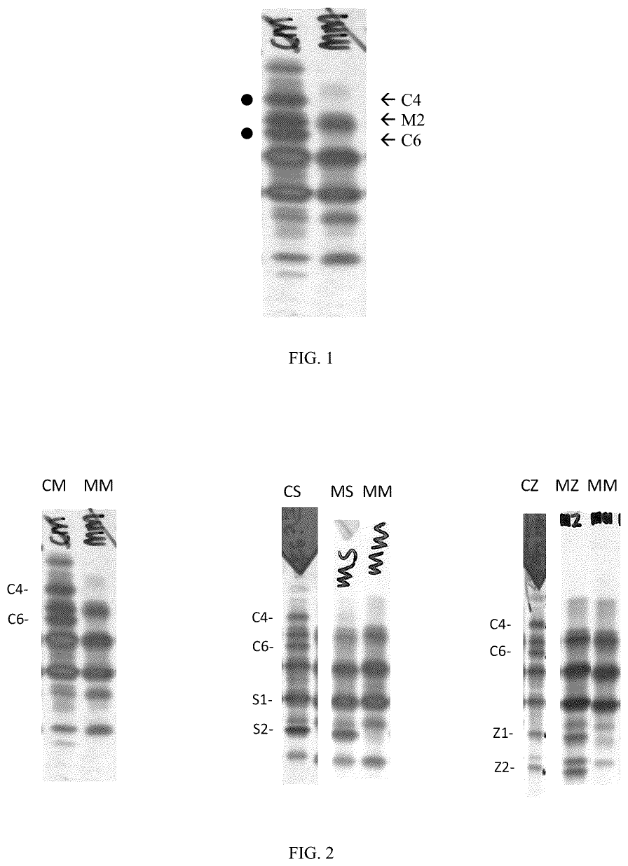

The CM phenotype is distinctly different by virtue of two bands, designated C4 and C6, anodal and cathodal to the M2 band of the M protein pattern on immunoelectrophoresis (IEF) gels. These are similarly present on gels that are phenotyped as CS and CZ. Referring to FIG. 1, the C4 and C6 are designated by arrows and dots that lic above the larger M2 (designated by arrow with rectangle) band that situated between them on adjacent CM and MM samples on IEFagarose gels. FIG. 2 demonstrates the correlating gels for CM compared to MM, CS compared to MS and CZ compared to MZ. The left gel shows CM with C4 and C6 bands adjacent to MM. The center gel shows CS with C4 and C6 bands adjacent to MS with S1 and S2 bands distinguishing MS and CS from MM. The right gel shows CZ with C4 and C6 bands adjacent to MZ with Z1 and Z2 bands distinguishing it from MM.

Any current (or future) test for determining whether a patient is infected with the SARS-COV-2 virus is (or will be) suitable for use in the invention. Suitable tests include but are not limited to an immunoassay for detecting nucleocapsid protein antigen from SARS-CoV-2 in samples (e.g., BINAXNOW from ABBOTT, BD VERITOR SARS-COV-2 from BECTON DICKINSON), a molecular diagnostic test utilizing nucleic acid amplification, such as RT-PCR, to detect viral RNA (e.g., METRIX COVID-19 TEST from APTITUDE MEDICAL SYSTEMS INC., CUE COVID-19 TEST from CUE HEALTH INC.).

The therapeutic method of the invention comprises administering AAT to patients infected with SARS-COV-2 and having the C phenotype. AAT is preferably administered intravenously in a therapeutically effective amount. In preferred embodiments, AAT is administered intravenously on a weekly basis in an amount from 10-1000 mg/kg of bodyweight (e.g., 10, 20, 30, 40, 50, 60, 70, 80, 90, 100, 110, 120, 130, 140, 150, 175, 200, 225, 250, 500, 750 or 1000 mg/kg).

AAT is preferably administered as early as possible after SARS-COV-2 infection. According to the NIH's “Clinical Spectrum of SARS-COV-2 Infection” (Sep. 26, 2022 update), “severe illness” is present in individuals who have SpO₂<94% on room air at sea level, a ratio of arterial partial pressure of oxygen to fraction of inspired oxygen (PaO2/FiO2) <300 mm Hg, a respiratory rate >30 breaths/min, or lung infiltrates >50%. Thus, in certain embodiments of the invention, AAT is first administered to the patient while the patient has an oxygen saturation level of at least 94% or at least 95% or at least 96% or 94-100% or 95-100%.

Additionally or alternatively, patients having the C phenotype can be administered medications indicated for COVID-19 patients likely to develop severe symptoms. Such medications include but are not limited to Baricitinib, Remdesivir, Nirmatrelvir, Ritonavir, Bebtelovimab and Molnupiravir.

### Kit

The invention also encompasses a kit for practicing one or more methods of the invention. The kit preferably comprises a first sample collection container for holding a sample to be tested for a SARS-COV-2 infection; and a second sample collection container for holding a sample to be tested for alpha-1-antitrypsin phenotype C. The sample collection containers can be the same or different. Either or both can be solely for the purpose of holding a sample or can be designed for multiple functions. Thus, for example, collection container(s) can be configured for mixing reagents with samples and for dispensing same.

In certain embodiments, the kit can further comprise at least one of an antigen-binding moiety effective to specifically bind to a SARS-COV-2 antigen.

In certain embodiments, the kit can further comprise a SARS-COV-2 specific antibody and a control antibody immobilized on two distinct locations of a substrate.

In certain embodiments, the kit can further comprise at least one of instructions for using the kit, swabs, reagents, buffers and test strips.

The invention will be illustrated in more detail with reference to the following Examples, but it should be understood that the present invention is not deemed to be limited thereto.

### EXAMPLES

### Study Design

AAT phenotyping was performed on 528 consecutive hospitalized unvaccinated PCR confirmed COVID patients (12). Phenotypes were performed using IEF through Quest Diagnostics, Inc. (San Juan Capistrano, California). Admission days were number of days from date of admission entry to date of discharge. Ventilator days were number of days from intubation to day of extubation. We defaulted to the highest level of ventilation necessary on any given day (e.g., if nasal cannula and high flow nasal cannula (HFNC) were employed on a given day it was designated HFNC). IL-6, HS-CRP, Alpha-1 antitrypsin serum level, Alpha-1 antitrypsin phenotype were drawn in the first 24 hours of admission. 30-day and 60-day respiratory and all-cause mortality and 30-day re-admission rates were primary endpoints. Respiratory death was defined as respiratory disease as a major or primary cause of death. All-cause was respiratory plus any other cause. 30-day and 60-day mortality was death within the first 30 or 60 days, respectively. Secondary endpoints included AAT, HS-CRP, IL-6 levels, IL-6/AAT ratio within 24 hours of admission, LOS, NIVV days, ICU days, and ventilator days. A two-week interval to determine phenotype resulted in ad hoc identification of deficiency alleles, C phenotype, and control MM.

### Immunoelectrophoresis

IEF at Quest Diagnostics, Inc. (San Juan Capistrano, California) employing Sebia Hydragel 18 A1AT Isofocusing kit employing agarose gels (Sebia Inc, Norcross, Georgia). Patient protein samples were separated to isoelectric point (pl) followed by incubation with peroxidase labeled anti-AlAT antisera, processed to remove unbound protein and then stained. The resulting banding pattern of each sample was used to determine the patient's A1AT phenotype.

### Next Generation Sequencing

Next generation sequencing for the Serpinal gene locus was performed at the Alpha-1 Center (Salt Lake City, Utah). Sample DNA was purified with an Auto Mate Express Instrument (Applied Biosystems; Waltham, Massachusetts). After coding exons 2-5 were amplified they were cycle sequenced with Big Dye® Terminator v3.1 (Thermo Fisher Scientific; Waltham, Massachusetts) and capillary electrophoresis on the Applied Biosystems 3500 Genetic Analyzer (Applied Biosystems). Sequence analysis employed Variant Reporter™ software (Thermo Fisher Scientific). Any identified abnormalities would have been compared to information available through the National Center for Biotechnology Information (NCBI). Selectively employed to confirm genotype of CM samples.

### Statistical Analysis

Test and control groups were compared using Chi-square tests of Independence and Odds Ratio with a 95% confidence interval for each of the conditions listed was calculated. Groups were compared using Welch's independent samples t-tests on numeric outcomes. A multiple regression model specifying mortality as the outcome variable was employed to assess mortality prediction.

### Results

448 MM, 44 CM and 36 deficiency alleles were identified (28 MS, 7 MZ, one SZ). Individuals identified as CM had characteristic C4 and C6 bands anodal and cathodal to the M2 band. Individuals identified as MM, MS, MZ had typical banding patterns for those phenotypes and lacked C4 and C6 bands.

### Deficiency Allele Versus MM Comparison

30- and 60-day respiratory mortality was higher in individuals with a deficiency allele than with those carrying an MM genotype (19.4 v 9.2%, p<0.05, OR=2.40 and 22.2 v 9.8%, p<0.05, OR=2.33). However, 30- and 60-day all-cause mortality did not achieve statistical significance in individuals with a deficiency allele compared to those with an MM allele. Thirty-day re-admission rates were much higher in individuals with any deficiency allele compared to control MM individuals (11.1 v 2.0%, p<0.001, OR=6.10) and MS versus MM (15.4 v 2.2%, p<0.001, OR=7.95). As expected, the acute AAT level were higher for MM versus a deficiency allele (196+51.9 v 153+46.2 mg/dl, p<0.001). There was no significant difference in hs-CRP, IL-6, or IL-6/AAT ratio between individuals with a deficiency alleles versus an MM allele. There was also no significant difference in LOS, days of NIVV, ICU days, or days on mechanical ventilation between the various alleles.

### CM Phenotype Versus MM Comparison

30- and 60-day respiratory mortality was higher for individuals with a CM phenotype versus MM (31.8 v 9.2%, p<0.001, OR=4.63, and 31.8 v 9.8%, p<0.001, OR=4.28). All-cause 30- and 60-day was higher for CM versus MM (40.9 v 11.2%, p<0.001, OR=5.51 and 40.9 v 12.1%, p<0.001, OR=5.05). 30-day re-admission rates were higher for CM than MM but not significant (4.5 v 2.0%, p=0.277, OR=2.32). Acute AAT levels were nearly identical for CM and MM (191+45.5 v 196+51.9 mg/dl) individuals and HS-CRP levels were significantly higher in CM versus MM individuals (14.3 v 9.72 mg/dl, p=0.001). The IL-6 levels and IL-6/AAT ratio were higher in CM than MM but did not achieve significance. LOS. ICU days and ventilator days were higher for CM than MM but also did not achieve significance. There was no difference for patients in days on NIVV.

### Combined Test Group Versus MM Comparison

The 80 patients who exhibited either a deficiency allele or the CM phenotype had significantly higher 30- and 60-day respiratory mortality than those carrying an MM phenotype (26.3 v 9.2%, p<0.001, OR=3.53 and 27.5 v 9.8%, p<0.001, OR=3.48). All-cause 30- and 60-day mortality was higher for the combined test group versus MM (31.3 v 11.2%, p<0.001, OR=3.62 and 32.5 v 12.1%, p<0.001, OR=3.51). Acute AAT levels were lower in the combined test group versus MM individuals(173+49.5 v 196+51.9 mg/DL, p<0.001). HS-CRP trended toward higher levels in the Test Group versus MM (11.4 v 9.72 mg/DL, p=0.075). There was no significant difference between the test group and MM controls for IL-6 serum levels or IL-6/AAT ratio. LOS trended towards significant for the Test Group versus MM (10.2 v 8.30 days, p=0.068). There were no significant differences between the test and control group phenotypes for ICU, ventilator, or NIVV days.

### Other Factors Affecting Mortality

Mean IL-6/AAT ratios were higher for all patients with 30-day and 60-day respiratory and all-cause mortality compared to survivors but did not achieve significance. In a multiple regression model for mortality the two major co-morbidities that predicted mortality were acute renal disease (OR=5.393, p<0.001) and prior Immunosuppression (OR=3.351, p=0.011). Prior cardiac pathology, hypertension, diabetes mellitus, prior respiratory disease, cancer, neurologic or psychiatric disease, and chronic renal disease while present in many admitted patients did not independently predict mortality. We also found no significant effect for sex, age or BMI (See Table 1 below for summary).

TABLE 1
Model Coefficients - 60 Day Respiratory Mortality

| Predictor | Estimate | SE | Z | p | Odds ratio |
|---|---|---|---|---|---|
| Intercept | 4.56285 | 1.5778 | 2.892 | 0.004 | 95.856 |
| No Cardiac Pathology − Cardiac | −0.08233 | 0.301 | −0.2735 | 0.784 | 0.921 |
| Pathology | | | | | |
| No Hypertension − Hypertension | −0.01929 | 0.3288 | −0.0587 | 0.953 | 0.981 |
| Not Diabetic − Diabetic | 0.38925 | 0.3148 | 1.2366 | 0.216 | 1.476 |
| No Respiratory Pathology − | 0.11702 | 0.3039 | 0.3851 | 0.7 | 1.124 |
| Respiratory Pathology | | | | | |
| No Cancer − Cancer | 0.3541 | 0.3772 | 0.9389 | 0.348 | 1.425 |
| No Neuropsychiatric Disorder − | −0.55477 | 0.4534 | −1.2235 | 0.221 | 0.574 |
| Neuropsychiatric Disorder | | | | | |
| No Chronic Renal Disease − Chronic | −0.52192 | 0.5268 | −0.9907 | 0.322 | 0.593 |
| Renal Disease | | | | | |
| No Acute Renal Disease − Acute | 1.68516 | 0.4684 | 3.5978 | <.001 | 5.393 |
| Renal Disease | | | | | |
| No Immunosuppression − | 1.2094 | 0.4755 | 2.5436 | 0.011 | 3.351 |
| Immunosuppression | | | | | |
| Male − Female | −0.3405 | 0.3003 | −1.1338 | 0.257 | 0.711 |
| AGE | −0.06486 | 0.0127 | −5.095 | <.001 | 0.937 |
| BMI | −0.00279 | 0.0211 | −0.1321 | 0.895 | 0.997 |

CM Sample Sequencing

Five CM samples submitted for next generation sequencing have MM genotype.

### Discussion

The studies were undertaken to confirm whether functional AAT is critical to inhibiting and resolving the inflammatory response to SARS-COV-2 such that individuals with deficiency states would exhibit worse prognoses. The most disadvantaged patients are likely the severely deficient PIZZ individuals, but these are rare. Relatively deficient patients like heterozygotes are altogether more common and frequently undiagnosed. In our series of 528 patients, we identified 36 (6.8%) individuals meeting the criteria. Even with this relative deficiency, the study described above demonstrated an increase in 30- and 60-day respiratory mortality. All-cause mortality at 30 and 60 days was short of reaching statistical significance. All this occurred without any significant difference in inflammation as measured by hs-CRP, IL-6 serum levels, or IL-6/AAT ratios. The presence of a deficiency allele also appears to predict an increased risk of re-admission, which is possibly attributable to expected delay in resolution of the inflammatory state due to the AAT deficiency.

More striking are the findings with the disease altered CM phenotype. These are genotypically MM individuals with the MM protein having a different banding pattern on IEF gels (13). Ordinarily, in any series this phenotype would be less than 1% (14). However, interestingly in our series of hospitalized patients we identified an increased number of patients (8.3%) with this phenotype. These patients had higher hs-CRP levels on admission than those identified as MM suggesting a higher inflammatory response in this patient population. We have previously identified this AAT phenotype in a variety of inflammatory conditions (13) and noted that it can resolve or occur across serial admissions (2). When present in our acute Covid patients the CM phenotype was associated with increased 30-day respiratory (Odds Ratio 4.63) and 60-day (Odds Ratio 4.28) mortality and 30-day all cause (Odds Ratio 5.51) and 60-day all cause (Odds Ratio 5.05) mortality. The effect was large enough that when CM patients were pooled with those with deficiency alleles all of those same 30- and 60-day mortality statistics were significant. Other endpoints pertaining to utilization like LOS, ICU days and ventilator days were also higher in CM patients but without statistical significance likely a reflection of the small sample size.

In prior studies, we have argued that the CM phenotype most likely represents a post-translational modification in glycosylation (13). Alterations in glycosylation of acute phase proteins in both chronic and acute conditions have been well described (15). Glycosylation has been noted to be affected by a variety of inflammatory conditions including HIV infection, malignancy, rheumatoid arthritis, sepsis and pancreatitis (16). Our published studies noted an association between the CM phenotype and cancer (13). In multivariate analysis of these SARS-2 COVID patients, cancer was not a factor identified with increased mortality. We infer that the increased mortality in our COVID patients identified as CM is independent of the presence of cancer. We have found that the AAT levels are almost identical in our MM and CM patients with COVID. Given the significant difference in mortality, we hypothesize that the CM phenotype would exhibit functional deficiency as an anti-protease and is likely clinically inactive.

In view of our findings, AAT phenotyping should be considered as a standard tool in constructing a plan of care and predicting outcome in SARS COV-2 patients. This approach identifies patients requiring more aggressive management and perhaps serve as an additional guideline for administration of Baricitinib or other therapies with demonstrated efficacy reserved for the sickest patients. Given that the delay in phenotypic analysis was only identified ad hoc, more rapid phenotype determination is preferred. The presence of a deficiency allele or CM phenotype could serve as indication for supplemental AAT therapy or therapies targeting normalization of glycosylation patterns in AAT. Furthermore, CM deserves potential consideration as a biomarker.

In conclusion, AAT phenotypes in hospitalized COVID patients revealed significant differences in 30- and 60-day respiratory mortality, all cause 30 and 60-day respiratory mortality and re-admission rates. 30- and 60-day respiratory mortality was higher in patients with a deficiency allele or the post-translationally modified CM phenotype. All-cause 30 and 60-day mortality was higher with the CM phenotype and the combined Test Group. Hospital re-admission rates were significantly higher in those with a deficiency allele. The presence of a relative deficiency in AAT as the result of a single deficiency allele does appear to contribute to adverse outcomes. More significantly, post-translationally modified AAT in the form of the CM phenotype portends a worse prognosis. The CM phenotype appears to be the ordinary AAT MM protein with reduced or absent function. Rapid AAT phenotyping could be of assistance in triaging ER patients to provide early, aggressive therapy in susceptible populations.

While the invention has been described in detail and with reference to specific examples thereof, it will be apparent to one skilled in the art that various changes and modifications can be made therein without departing from the spirit and scope thereof.

### REFERENCES CITED

1. Abdulkarim et al. Alpha 1 Antitrypsin Mutation. [Updated 2022 Sep 26]. In: StatPearls [Internet]. Treasure Island (FL): StatPearls Publishing; 2022 Jan-. Available from: https://www.ncbi.nlm.nih.gov/books/NBK482180/.
2. Law et al. “Variable expression of the PiC phenotype across successive admissions.” In TP1. TP001 MECHANISTIC AND TRANSLATIONAL STUDIES IN COPD, pp.

A1228-A1228. American Thoracic Society, 2021.
3. Oguntuyo et al. In plain sight. The role of alpha-1 antitrypsin in COVID 19 pathogenesis and therapeutics. Bio RXIV 2020 Aug. doi.10.1101/2020.08.14.248880.
4. Azouz et al. Alpha-1 antitrypsin is an inhibitor of the SARS-COV-2-priming protease TMPRSS2. Pathog Immun 2021. 6 (1): 55-74.
5. McElvaney et al. Characterization of the inflammatory response to severe COVID-19 Illness. Am J Resp Crit Care 2020 June. 202 (6): 812-821.
6. Yang et al. Alpha-1 antitrypsin for COVID-19 treatment: dual role in antiviral infection and anti-inflammation. Front Pharmacol 2020 Dec. 11:615398.
7. Ritzmann et al. Therapeutic application of alpha-1 antitrypsin in COVID-19. Am J Respir Crit Care Med 2021 July. 204 (2): 224-227.
8. Law et al. Clinical disease associations with alpha-1 antitrypsin PiCM phenotype. Conference paper Amer Thor Soc 2021 Int Conf 2021.
9. Robinet-Levy, et al. “Techniques d′identification des groupes Pi: Premières statistiques languedociennes.” Revue Française de Transfusion 15, no. 1 (1972): 61-72.
10. Greene et al. “AAT Phenotype Identification by Isoelectric Focusing.” In Alpha-1 Antitrypsin Deficiency, pp. 33-44. Humana Press, New York, NY, 2017.
11. Greene et al. “Facilitating the laboratory diagnosis of al-antitrypsin deficiency.” American journal of clinical pathology 139, no. 2 (2013): 184-191.
12. Law et al. (12/15/2021). Hospitalized non-vaccinated COVID Patients with AAT Deficiency alleles or Disease Altered CM phenotype have and all-higher 30 and 60-day respiratory cause mortality. https://www.researchgate.net/publication/357097710_Hospitalized_non-vaccinated_COVID_Patients_with_AAT_Deficiency_alleles_or_Disease_Altered_C M_phenotype_have_higher_30_and_60-day_respiratory_and_all-_cause_mortality.
13. Law et al. Clinical disease associations with alpha-1 antitrypsin PiCM phenotype. Conference paper Amer Thor Soc 2021 Int Conf 2021.
14. Bornhorst et al. Alpha1-antitrypsin phenotypes and associated serum protein concentrations in a large clinical population. Chest 2013. 143 (4): 1000-1008.
15. McCarthy et al. The role and importance of glycosylation of acute phase proteins with focus on alpha-1 antitrypsin in acute and chronic inflammatory conditions. J Proteome Res 2014. 13 (7): 3131-3143.
16. Marino et al. Changes in serum in serum N-glycosylation profiles: functional significance and potential for diagnostics. Carbohydrate Chemistry: Chemical and Biological Approaches. Rauter, AP, Ed. RSC Publishing: London, 2012.

What is claimed is:

1. A method of treating a patient infected with SARS-COV-2 virus, said method comprising:
phenotyping a sample obtained from the patient; and
administering alpha-1-antitrypsin to the patient only if an alpha-1-antitrypsin C phenotype is detected in the sample,
wherein the alpha-1-antitrypsin is effective to treat the patient infected with the SARS-CoV-2 virus.

2. The method of claim 1, wherein the phenotyping comprises the sequential steps of:
separating proteins in the sample by isoelectric focusing;
incubating the sample with peroxidase labeled anti-alpha-1-antitrypsin antisera;
removing unbound proteins;
staining bound proteins; and
reading a resulting banding pattern to determine whether the patient has the alpha-1-antitrypsin C phenotype.

3. The method of claim 1, wherein the alpha-1-antitrypsin is administered intravenously.

4. The method of claim 1, wherein the alpha-1-antitrypsin is first administered to the patient while the patient has an oxygen saturation level of at least 94%.

5. The method of claim 1, further comprising administering to the patient at least one member selected from the group consisting of Baricitinib, Remdesivir, Nirmatrelvir, Ritonavir, Bebtelovimab and Molnupiravir.

6. The method of claim 1, further comprising detecting whether the patient is infected with SARS-COV-2 virus.

7. The method of claim 1, wherein the alpha-1-antitrypsin C phenotype is CM, CS or CZ phenotype.

8. The method of claim 2, wherein the alpha-1-antitrypsin is administered intravenously.

9. The method of claim 8, wherein the alpha-1-antitrypsin is first administered to the patient while the patient has an oxygen saturation level of at least 94%.

10. The method of claim 9, further comprising administering to the patient at least one member selected from the group consisting of Baricitinib, Remdesivir, Nirmatrelvir, Ritonavir, Bebtelovimab and Molnupiravir.

11. The method of claim 10, further comprising detecting whether the patient is infected with SARS-COV-2 virus.

12. The method of claim 11, wherein the alpha-1-antitrypsin C phenotype is CM, CS or CZ phenotype.

13. A kit configured to detect a SARS-COV-2 infection and an alpha-1-antitrypsin C phenotype, said kit comprising:
a first sample collection container for holding a sample to be tested for a SARS-COV-2 infection; and
a second sample collection container for holding a sample to be tested for alpha-1-antitrypsin phenotype C.

14. The kit of claim 13, further comprising an antigen-binding moiety effective to specifically bind to a SARS-COV-2 antigen.

15. The kit of claim 13, further comprising a SARS-COV-2 specific antibody and a control antibody immobilized on two distinct locations of a substrate.
